(19) United States
(12) Patent Application Publication
(10) Pub. No.: US 2020/0000742 A1
(43) Pub. Date: Jan. 2, 2020

(54) COMBINATION

(71) Applicant: HELPERBY THERAPEUTICS LIMITED, London (GB)

(72) Inventors: Anthony COATES, London (GB); Yanmin HU, London (GB)

(21) Appl. No.: 16/334,002

(22) PCT Filed: Oct. 5, 2017
(86) PCT No.: PCT/GB2017/053022

§ 371 Date: Mar. 16, 2019

(30) Foreign Application Priority Data
Oct. 5, 2016 (GB) 1616909.6

(51) Int. Cl. A61K 31/055; A61K 31/382; A61K 31/5415; A61K 31/197; A61K 31/407; A61P 31/04

(52) U.S. Cl. CPC A61K 31/055; A61K 31/382; A61P 31/04; A61K 31/197; A61K 31/407; A61K 31/5415

(57) ABSTRACT

The invention provides a combination comprising at least one compound selected from dichlorophen, chlorprothixene, perphenazine, thioridazine, trifluoperazine and edetate or a pharmaceutically acceptable derivative thereof and a carbapenem or a pharmaceutically acceptable derivative thereof. This combination is particularly useful for the treatment of microbial infections.

### CROSS-REFERENCE TO RELATED APPLICATION

This application is a 35 U.S.C. § 371 national stage filing of PCT Application No. PCT/GB2017/053022 filed on Oct. 5, 2017, which claims priority to Great Britain Patent Application No. 1616909.6 filed on Oct. 5, 2016, each of which are incorporated herein in their entirety by reference.

### FIELD OF THE INVENTION

The present invention relates to the combination of at least one compound selected from dichlorophen, chlorprothixene (e.g. chlorprothixene hydrochloride), perphenazine, thioridazine (e.g. thioridazine hydrochloride), trifluoperazine (e.g. trifluoperazine hydrochloride) and edetate (e.g. edetate disodium) or a pharmaceutically acceptable derivative thereof with a carbapenem or a pharmaceutically acceptable derivative thereof, and the use of this combination for the treatment of microbial infections. In particular, it relates to the use of such combinations to kill multiplying or clinically latent microorganisms associated with microbial infections, e.g. Gram-negative bacterial infections.

### BACKGROUND

Carbapenems are antibiotics used for the treatment of infections known or suspected to be caused by multidrug-resistant (MDR) bacteria. Like penicillin and cephalosporin, they are members of the beta lactam class of antibiotics, which kill bacteria by binding to penicillin-binding proteins and inhibiting cell wall synthesis. Unlike cephalosporins and penicillin, however, the carbapenems exhibit a broad spectrum of activity and have greater potency again Gram-positive and Gram-negative bacteria. They are therefore often used as a last resort when patients with infections become severely ill or are suspected of harboring resistant bacteria (Antimicrob. Agents Chemother., 55, 4943-4960 (2011)). Unfortunately, however, several studies show that resistance to carbapenems is increasing throughout the world.

Carbapenems which have been approved for clinical use include imipenem, meropenem, ertapenem, doripenem, panipenem/betamipron and biapenem. Other carbapenems include razupenem, tebipenem, lenapenem, tomopenem and thienpenem. Meropenem is marketed under the trade names Optinem (meropenem trihydrate) and Meronem (meropenem trihydrate or meropenem) in Europe.

In view of the problem of bacterial resistance and the importance of antimicrobial agents such as carbapenems in the fight against bacterial infection, the identification of compounds capable of enhancing the antimicrobial activity of such agents addresses an important need.

International Patent Application published as WO2000/028074 describes a method of screening compounds to determine their ability to kill log phase (i.e. multiplying) and/or clinically latent microorganisms. Using this method, the Applicant has observed that many known compounds, including dichlorophen, chlorprothixene (e.g. chlorprothixene hydrochloride), perphenazine, thioridazine (e.g. thioridazine hydrochloride), trifluoperazine (e.g. trifluoperazine hydrochloride) and edetate (e.g. edetate disodium) or pharmaceutically acceptable derivatives thereof, have a synergistic effect with carbapenems or pharmaceutically acceptable derivatives thereof, such as meropenem (e.g. meropenem trihydrate), against multiplying and/or clinically latent microorganisms.

The present invention is based on the unexpected finding that the combination of at least one compound selected from dichlorophen, chlorprothixene, perphenazine, thioridazine, trifluoperazine and edetate or a pharmaceutically acceptable derivative thereof, and a carbapenem or a pharmaceutically acceptable derivative thereof (e.g. meropenem or a pharmaceutically acceptable derivative thereof) exhibits synergistic antimicrobial activity against log phase (i.e. multiplying) and/or clinically latent microorganisms. Particularly against log phase bacteria. In other words, the combination has a greater biological activity than the expected additive effect of each agent at the stated dosage level. The surprising biological activity of the combinations of the present invention offers the opportunity to shorten chemotherapy regimens and may result in a reduction in the emergence of microbial resistance associated with the use of such combinations.

Synergy in the context of antimicrobial drugs is measured in a number of ways that conform to the generally accepted opinion that “synergy is an effect greater than additive”. One of the ways to assess whether synergy has been observed is to use the “chequerboard” technique. This is a well-accepted method that leads to the generation of a value called the fractional inhibitory concentration index (FICI). Orhan et al J. Clin. Microbiol. 2005, 43(1):140 describes the chequerboard method and analysis in the paragraph bridging pages 140-141, and explains that the FICI value is a ratio of the sum of the MIC (Minimum Inhibitory Concentration) level of each individual component alone and in the mixture. The combination is considered synergistic when the ΣFIC is ≤0.5, indifferent when the ΣFIC is >0.5 but <2, and antagonistic when the ΣFIC is ≥2.

Another accepted test for ascertaining the presence or absence of synergy is to use time-kill methods. This involves the dynamic effect of a drug combination being compared to each drug alone when assessing the effect on bacterial log or stationary-growth over time. Again, the possible results are for synergistic, additive or antagonistic effects.

### SUMMARY OF THE INVENTION

Thus, in one embodiment the present invention provides a combination of at least one compound selected from dichlorophen, chlorprothixene, perphenazine, thioridazine, trifluoperazine and edetate or a pharmaceutically acceptable derivative thereof and a carbapenem or a pharmaceutically acceptable derivative thereof.

Preferably the carbapenem is meropenem, imipenem, doripenem or ertapenem or a pharmaceutically acceptable derivative thereof. More preferably the carbapenem is meropenem or a pharmaceutically acceptable derivative thereof, e.g. meropenem trihydrate.

Preferably the compound is selected from dichlorophen, chlorprothixene hydrochloride, perphenazine, thioridazine hydrochloride, trifluoperazine and edetate disodium. More preferably the compound is edetate disodium.

The invention therefore provides:

a combination of dichlorophen or a pharmaceutically acceptable derivative thereof and a carbapenem or a pharmaceutically acceptable derivative thereof (e.g. meropenem or a pharmaceutically acceptable derivative thereof).

a combination of chlorprothixene or a pharmaceutically acceptable derivative thereof and a carbapenem or a pharmaceutically acceptable derivative thereof (e.g. meropenem or a pharmaceutically acceptable derivative thereof).

a combination of perphenazine or a pharmaceutically acceptable derivative thereof and a carbapenem or a pharmaceutically acceptable derivative thereof (e.g. meropenem or a pharmaceutically acceptable derivative thereof).

a combination of thioridazine or a pharmaceutically acceptable derivative thereof and a carbapenem or a pharmaceutically acceptable derivative thereof (e.g. meropenem or a pharmaceutically acceptable derivative thereof).

a combination of trifluoperazine or a pharmaceutically acceptable derivative thereof and a carbapenem or a pharmaceutically acceptable derivative thereof (e.g. meropenem or a pharmaceutically acceptable derivative thereof).

a combination of edetate or a pharmaceutically acceptable derivative thereof and a carbapenem or a pharmaceutically acceptable derivative thereof (e.g. meropenem or a pharmaceutically acceptable derivative thereof).

In another embodiment the present invention provides the use of the combinations defined hereinabove, in the manufacture of a medicament for treating a microbial infection.

Additionally the present invention provides the combinations defined hereinabove, for use in the treatment of a microbial infection, preferably for use in the treatment of a bacterial infection.

In a further embodiment, the invention provides a method of treating a microbial infection which comprises administering to a mammal, including man, at least one of the combinations defined hereinabove.

There is also provided a pharmaceutical composition comprising at least one compound selected from dichlorophen, chlorprothixene, perphenazine, thioridazine, trifluoperazine and edetate or a pharmaceutically acceptable derivative thereof in combination with a carbapenem or a pharmaceutically acceptable derivative thereof, and a pharmaceutically acceptable adjuvant, diluent or carrier. In one embodiment the pharmaceutical composition is for use in the treatment of a microbial infection, preferably wherein the microbial infection is a bacterial infection.

In a further embodiment, the invention relates to a product comprising at least one compound selected from dichlorophen, chlorprothixene, perphenazine, thioridazine, trifluoperazine and edetate or a pharmaceutically acceptable derivative thereof and a carbapenem or a pharmaceutically acceptable derivative thereof, as a combined preparation for simultaneous, separate or sequential use in killing multiplying and/or clinically latent microorganisms associated with a microbial infection. Preferably for killing multiplying bacteria associated with a bacterial infection.

### BRIEF DESCRIPTION OF THE DRAWINGS

FIG. 1 is a time-kill curve (Log CFU/ml against time (hours)) showing the antimicrobial activity of meropenem trihydrate at a concentration of 512 mg/L, dichlorophen at a concentration of 64 mg/L and the combination of meropenem trihydrate (512 mg/L) and dichlorophen (64 mg/L) against log phase NDM-1 K. pneumoniae.

### DETAILED DESCRIPTION OF THE INVENTION

As described below, the combinations of the present invention have been demonstrated to be particularly effective against drug-resistant bacteria, particularly drug-resistant Gram-negative bacteria, opening the way for said combinations to be administered both to drug-resistant strains and in said strains before drug-resistance is built up, i.e. as a first line treatment.

As used herein, the term “in combination with” covers both separate and sequential administration of the compound and the carbapenem or pharmaceutically acceptable derivatives thereof. When the compound and the carbapenem are administered sequentially, either the compound or the carbapenem may be administered first. When administration is simultaneous, the compound and the carbapenem may be administered either in the same or a different pharmaceutical composition. Adjunctive therapy, i.e. where one agent is used as a primary treatment and the other agent is used to assist that primary treatment, is also an embodiment of the present invention.

The combinations of the present invention may be used to treat microbial infections. In particular they may be used to kill multiplying and/or clinically latent microorganisms associated with microbial infections, preferably multiplying microorganisms associated with microbial infections, e.g. multiplying bacteria associated with Gram-negative bacterial infections. References herein to the treatment of a microbial infection therefore include killing multiplying and/or clinically latent microorganisms associated with such infections.

As used herein, “kill” means a loss of viability as assessed by a lack of metabolic activity.

As used herein, “clinically latent microorganism” means a microorganism that is metabolically active but has a growth rate that is below the threshold of infectious disease expression. The threshold of infectious disease expression refers to the growth rate threshold below which symptoms of infectious disease in a host are absent.

The metabolic activity of clinically latent microorganisms can be determined by several methods known to those skilled in the art; for example, by measuring mRNA levels in the microorganisms or by determining their rate of uridine uptake. In this respect, clinically latent microorganisms, when compared to microorganisms under logarithmic growth conditions (in vitro or in vivo), possess reduced but still significant levels of:
(I) mRNA (e.g. from 0.0001 to 50%, such as from 1 to 30, 5 to 25 or 10 to 20%, of the level of mRNA); and/or
(II) uridine (e.g. [³H]uridine) uptake (e.g. from 0.0005 to 50%, such as from 1 to 40, 15 to 35 or 20 to 30% of the level of [³H]uridine uptake).

Clinically latent microorganisms typically possess a number of identifiable characteristics. For example, they may be viable but non-culturable; i.e. they cannot typically be detected by standard culture techniques, but are detectable and quantifiable by techniques such as broth dilution counting, microscopy, or molecular techniques such as polymerase chain reaction. In addition, clinically latent microorganisms are phenotypically tolerant, and as such are sensitive (in log phase) to the biostatic effects of conventional antimicrobial agents (i.e. microorganisms for which the minimum inhibitory concentration (MIC) of a conventional antimicrobial is substantially unchanged); but possess drastically decreased susceptibility to drug-induced killing (e.g. microorganisms for which, with any given conventional antimicrobial agent, the ratio of minimum microbiocidal concentration (e.g. minimum bactericidal concentration, MBC) to MIC is 10 or more).

As used herein, the term “microorganisms” means fungi and bacteria. References herein to “microbial”, “antimicrobial” and “antimicrobially” shall be interpreted accordingly. For example, the term “microbial” means fungal or bacterial, and “microbial infection” means any fungal or bacterial infection.

In one embodiment of the invention, one or more of the aforementioned combinations is used to treat a bacterial infection, in particular the combinations may be used to kill multiplying and/or clinically latent microorganisms associated with a bacterial infection. As used herein, the term “bacteria” (and derivatives thereof, such as “microbial infection”) includes, but is not limited to, references to organisms (or infections due to organisms) of the following classes and specific types:

Gram-positive cocci, such as Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus); Streptococci (e.g. beta-haemolytic, pyogenic streptococci (such as Strept. agalactiae, Strept. canis, Strept dysgalactiae dysgalactiae, Strept dysgalactiae equisimilis, Strept equi equi, Strept equi zooepidemicus, Strept. iniae, Strept. porcinus and Strept pyogenes), microaerophilic, pyogenic streptococci (Streptococcus “milleri”, such as Strept. anginosus, Strept constellatus constellatus, Strept constellatus pharyngidis and Strept intermedius), oral streptococci of the “mitis” (alpha-haemolytic-Streptococcus “viridans”, such as Strept. mitis, Strept. oralis, Strept. sanguinis, Strept. cristatus, Strept gordonii and Strept. parasanguinis), “salivarius” (non-haemolytic, such as Strept salivarius and Strept vestibularis) and “mutans” (tooth-surface streptococci, such as Strept. criceti, Strept. mutans, Strept ratti and Strept. sobrinus) groups, Strept. acidominimus, Strept. bovis, Strept. faecalis, Strept. equinus, Strept pneumoniae and Strept. suis, or Streptococci alternatively classified as Group A, B, C, D, E, G, L, P, U or V Streptococcus);

Gram-negative cocci, such as Neisseria gonorrhoeae, Neisseria meningitidis, Neisseria cinerea, Neisseria elongate, Neisseria flavescens, Neisseria lactamica, Neisseria mucosa, Neisseria sicca, Neisseria subflava and Neisseria weaveri; Bacillaceae, such as Bacillus anthracis, Bacillus subtilis, Bacillus thuringiensis, Bacillus stearothermophilus and Bacillus cereus; Enterobacteriaceae, such as Escherichia coli, Enterobacter (e.g. Enterobacter aerogenes, Enterobacter agglomerans and Enterobacter cloacae), Citrobacter (such as Citrob. freundii and Citrob. divernis), Hafnia (e.g. Hafnia alvei), Erwinia (e.g. Erwinia persicinus), Morganella morganii, Salmonella (Salmonella enterica and Salmonella typhi), Shigella (e.g. Shigella dysenteriae, Shigella flexneri, Shigella boydii and Shigella sonnei), Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis), Proteus (e.g. Pr. mirabilis, Pr. rettgeri and Pr. vulgaris), Providencia (e.g. Providencia alcalifaciens, Providencia rettgeri and Providencia stuartii), Serratia (e.g. Serratia marcescens and Serratia liquifaciens), and Yersinia (e.g. Yersinia enterocolitica, Yersinia pestis and Yersinia pseudotuberculosis); Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius); Helicobacter (e.g. Helicobacter pylori, Helicobacter cinaedi and Helicobacter fennelliae); Acinetobacter (e.g. A. baumanii, A. calcoaceticus, A. haemolyticus, A. johnsonii, A. junii, A. Iwoffi and A. radioresistens); Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzen); Bacteroides fragilis; Peptococcus (e.g. Peptococcus niger); Peptostreptococcus; Clostridium (e.g. C. perfringens, C. difficile, C. botulinum, C. tetani, C. absonum, C. argentinense, C. baratii, C. bifermentans, C. beijerinckii, C. butyricum, C. cadaveris, C. camis, C. celatum, C. clostridioforme, C. cochlearium, C. cocleatum, C. fallax, C. ghonfi, C. glycolicum, C. haemolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tertium); Mycoplasma (e.g. M. pneumoniae, M. hominis, M. genitalium and M. urealyticum); Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium lentiflavum, Mycobacterium malmoense, Mycobacterium mucogenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium the thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi); Haemophilus (e.g. Haemophilus influenzae, Haemophilus ducreyi, Haemophilus aegyptius, Haemophilus parainfluenzae, Haemophilus haemolyticus and Haemophilus parahaemolyticus); Actinobacillus (e.g. Actinobacillus actinomycetemcomitans, Actinobacillus equuli, Actinobacillus hominis, Actinobacillus lignieresii, Actinobacillus suis and Actinobacillus ureae); Actinomyces (e.g. Actinomyces israelii); Brucella (e.g. Brucella abortus, Brucella canis, Brucella melintensis and Brucella suis); Campylobacter (e.g. Campylobacter jejuni, Campylobacter coli, Campylobacter lari and Campylobacter fetus); Listeria monocytogenes; Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus); Erysipelothrix rhusopathiae; Corynebacteriaceae (e.g. Corynebacterium diphtheriae, Corynebacterium jeikeum and Corynebacterium urealyticum); Spirochaetaceae, such as Borrelia (e.g. Borrelia recurrentis, Borrelia burgdorferi, Borrelia afzelii, Borrelia andersonfi, Borrelia bissettii, Borrelia garinfi, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. pertenue and Treponema carateum); Pasteurella (e.g. Pasteurella aerogenes, Pasteurella bettyae, Pasteurella canis, Pasteurella dagmatis, Pasteurella gaffinarum, Pasteurella haemolytica, Pasteurella multocida multocida, Pasteurella multocida gafficida, Pasteurella multocida septica, Pasteurella pneumotropica and Pasteurella stomatis); Bordetella (e.g. Bordetella bronchiseptica, Bordetella hinzii, Bordetella holmseii, Bordetella parapertussis, Bordetella pertussis and Bordetella trematum); Nocardiaceae, such as Nocardia (e.g. Nocardia asteroides and Nocardia brasiliensis); Rickettsia (e.g. Ricksettsii or Coxiella burnetii); Legionella (e.g. Legionalla anisa, Legionalla birminghamensis, Legionalla bozemanii), Legionalla cincinnatiensis, Legionalla dumoffii, Legionalla feelefi, Legionalla gormanii, Legionalla hackeliae, Legionalla israelensis, Legionalla jordanis, Legionalla lansingensis, Legionalla longbeachae, Legionalla maceachernii, Legionalla micdadei, Legionalla oakridgensis, Legionalla pneumophila, Legionalla sainthelensi, Legionalla tucsonensis and Legionalla wadsworthii); Moraxella catarrhalis; Cyclospora cayetanensis; Entamoeba histolytica; Giardia lamblia; Trichomonas vaginalis; Toxoplasma gondii; Stenotrophomonas maltophilia; Burkholderia cepacia; Burkholderia mallei and Burkholderia pseudomallei; Francisella tularensis; Gardnerella (e.g. Gardneralla vaginalis and Gardneralla mobiluncus); Streptobacillus moniliformis; Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena); Bartonella (Bartonella baciffiformis, Bartonella clarridgeiae, Bartonella elizabethae, Bartonella henselae, Bartonella quintana and Bartonella vinsonii arupensis); Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilii); Spirillium (e.g. Spirillum minus); Baceteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides uniformis, Bacteroides ureolyticus and Bacteroides vulgatus); Prevotella (e.g. Prevotella bivia, Prevotella buccae, Prevotella corporis, Prevotella dentalis (Mitsuokella dentalis), Prevotella denticola, Prevotella disiens, Prevotella enoeca, Prevotella heparinolytica, Prevotella intermedia, Prevotella loeschii, Prevotella melaninogenica, Prevotella nigrescens, Prevotella oralis, Prevotella oris, Prevotella oulora, Prevotella tannerae, Prevotella venoralis and Prevotella zoogleoformans); Porphyromonas (e.g. Porphyromonas asaccharolytica, Porphyromonas cangingivalis, Porphyromonas canons, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae); Fusobacterium (e.g. F. gonadiaformans, F. mortiferum, F. naviforme, F. necrogenes, F. necrophorum necrophorum, F. necrophorum fundiliforme, F. nucleatum nucleatum, F. nucleatum fusiforme, F. nucleatum polymorphum, F. nucleatum vincentii, F. periodonticum, F. russii, F. ulcerans and F. varium); Chlamydia (e.g. Chlamydia trachomatis); Cryptosporidium (e.g. C. parvum, C. hominis, C. canis, C. felis, C. meleagridis and C. muris); Chlamydophila (e.g. Chlamydophila abortus (Chlamydia psittaci), Chlamydophila pneumoniae (Chlamydia pneumoniae) and Chlamydophila psittaci (Chlamydia psittaci)); Leuconostoc (e.g. Leuconostoc citreum, Leuconostoc cremoris, Leuconostoc dextranicum, Leuconostoc lactis, Leuconostoc mesenteroides and Leuconostoc pseudomesenteroides); Gemella (e.g. Gemella bergeri, Gemella haemolysans, Gemella morbillorum and Gemella sanguinis); and Ureaplasma (e.g. Ureaplasma parvum and Ureaplasma urealyticum).

Preferably, the bacterial infections treated by the combinations described herein are Gram-negative bacterial infections. Particular Gram-negative bacteria that may be treated using a combination of the invention include:

Enterobacteriaceae, such as Escherichia coli, Klebsiella (e.g. Klebs. pneumoniae and Klebs. oxytoca) and Proteus (e.g. Pr. mirabilis, Pr. rettgeri and Pr. vulgaris); Haemophilis influenzae; Mycobacteria, such as Mycobacterium tuberculosis; and Enterobacter (e.g. Enterobacter cloacae).

Preferably, the bacteria are Enterobacteriaceae, such as Escherichia coli and Klebsiella (e.g. Klebs. pneumoniae and Klebs. oxytoca). Particularly preferred are Escherichia coli, and Klebs. pneumoniae (e.g. Klebs. pneumoniae subsp. pneumoniae).

In all embodiments it is preferable that the combination therapy is synergistic as compared to the administration of the combination components taken alone.

The combination of the present invention is particularly beneficial in treating (multi)-drug-resistant ((M)DR) bacteria. With respect to Enterobacteriaceae, drug resistance most often builds up to carbapenemase i.e. carbapenemase-resistant strains and “extended spectrum β-lactamase” (ESBL) strains for example New Delhi Metallo-beta-lactamase-1 (NDM-1) resistant Klebs. Pneumonia, and NDM-1 E. coli.

It should be kept in mind that although a combination such as that claimed may initially be demonstrated to be functional in treating (M)DR strains, they can then be used in treating non-resistant strains. This is especially valuable in the context of the presently claimed combination where the primary therapy for Enterobacteriaceae, such as Escherichia coli, and Klebsiella (e.g. Klebs. pneumoniae and Klebs. oxytoca) are antimicrobial drugs that are expensive due to prevailing patent protection. The replacement of such “ethical” drugs by a combination of “generic” antibiotics is thought to be beneficial from a therapeutic perspective as well as financial/economic perspective in times where governments are seeking to reduce the cost of healthcare.

The combinations of the present invention may be used to treat infections associated with any of the above-mentioned bacterial organisms, and in particular they may be used for killing multiplying and/or clinically latent microorganisms associated with such an infection, e.g. a Gram-negative bacterial infection.

Particular conditions which may be treated using the combination of the present invention include those which are caused by Gram-negative bacteria such as abscesses, asthma, bacilliary dysentry, bacterial conjunctivitis, bacterial keratitis, bacterial vaginosis, bone and joint infections, bronchitis (acute or chronic), brucellosis, burn wounds, cat scratch fever, cellulitis, chancroid, cholangitis, cholecystitis, cystic fibrosis, cystitis, nephritis, diffuse panbronchiolitis, dental caries, diseases of the upper respiratory tract, empymea, endocarditis, endometritis, enteric fever, enteritis, epididymitis, epiglottitis, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leptospirosis, listeriosis, liver abscesses, Lyme disease, lymphogranuloma venerium, mastitis, mastoiditis, meningitis and infections of the nervous system, non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, pleural effusion, pneumonia, postoperative wound infections, postoperative gas gangrene, prostatitis, pseudo-membranous colitis, psittacosis, pyelonephritis, Q fever, rat-bite fever, Ritter's disease, salmonellosis, salpingitis, septic arthritis, septic infections, septicameia, systemic infections, tonsillitis, trachoma, typhoid, urethritis, urinary tract infections, wound infections; or infections with, Escherichia coli, Klebs. pneumoniae, Klebs. oxytoca, Pr. mirabilis, Pr. rettgeri, Pr. vulgaris, Haemophilis influenzae, Enterococcus faecalis, Enterococcus faecium, and Enterobacter cloacae.

It will be appreciated that references herein to “treatment” extend to prophylaxis as well as the treatment of established diseases or symptoms.

As used herein the term “pharmaceutically acceptable derivative” means:

(a) pharmaceutically acceptable salts; and/or
(b) solvates (including hydrates).

Suitable acid addition salts include carbon/late salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfite, phosphate, monohydrogenphosphate, dihydrogenphosphate, metaphosphate, pyrophosphate or nitrate salts, and the like.

References herein to a carbapenem mean a compound with the following core chemical structure:

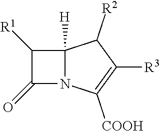

where R¹, R² and R³ are different substituents. The carbapenem may be selected from the group consisting of imipenem, meropenem, ertapenem, doripenem, panipenem, biapenem, razupenem, tebipenem, lenapenem, tomopenem and thienpenem or a pharmaceutically derivative thereof, e.g. meropenem trihydrate. These compounds have the following chemical structures, including the above chemical core.

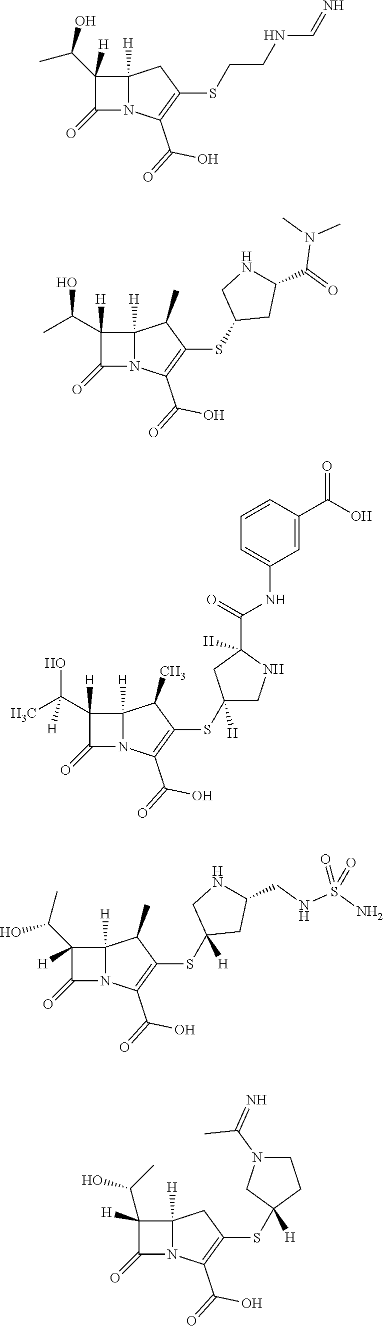

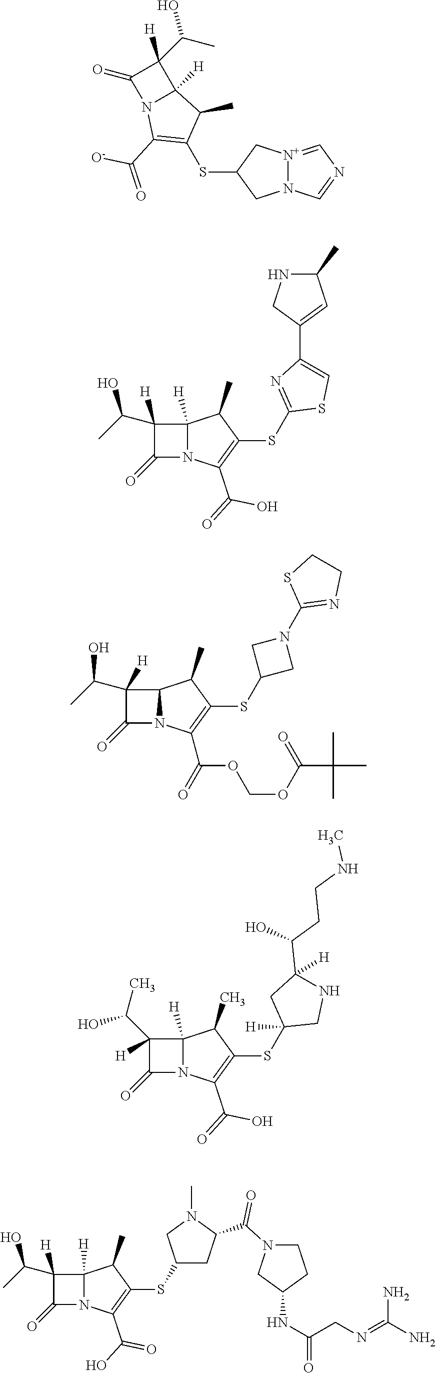

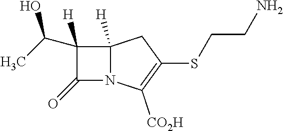 [SMILES: C[C@@H](O)[C@H]1C(=O)N2C(C(=O)O)=C(SCCN)C[C@H]12]

Preferably the carbapenem is selected from the group consisting of imipenem, meropenem, ertapenem, doripenem, panipenem and biapenem, or a pharmaceutically acceptable derivative thereof. More preferably the carbapenem is selected from meropenem, imipenem, doripenem and ertapenem or a pharmaceutically acceptable derivative thereof. Most preferably the carbapenem is meropenem or a pharmaceutically acceptable derivative thereof, e.g. meropenem trihydrate.

Dichlorophen is an anticestodal agent, which is typically used in combination with toluene for removing parasites from dogs and cats. It is also known as 4-chloro-2-[(5-chloro-2-hydroxyphenyl)methyl] phenol, dichlorophene, or bis(5-chloro-2-hydroxyphenyl)methane and has the following chemical structure:

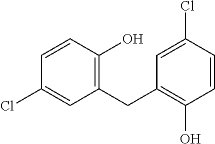 [SMILES: Oc1ccc(Cl)cc1Cc1cc(Cl)ccc1O]

Chlorprothixene is an antipsychotic drug of the thioxanthene class. It is also known as (Z)-3-(2-chlorothioxanthen-9-ylidene)-N,N-dimethyl-propan-1-amine, and has the following chemical structure:

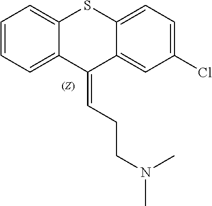 [SMILES: CN(C)CC/C=C1/c2ccccc2Sc2ccc(Cl)cc21]

Chlorprothixene may be used in the form of its hydrochloride salt. In the present invention it is preferably in salt form, i.e. as chlorprothixene hydrochloride.

Perphenazine is an antipsychotic drug which is chemically classified as piperazinyl phenothiazine and commercially available under the brand name Trilafon, Fentazin or Triptafen. It has the chemical name of 2-[4-[3-(2-chloro-10H-phenothiazin-10-yl) propyl]piperazin-1-yl]ethanol and the following chemical structure:

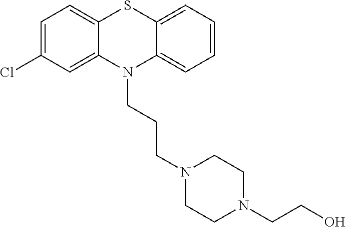 [SMILES: OCCN1CCN(CCCN2c3ccccc3Sc3ccc(Cl)cc32)CC1]

Perphenazine may be used in the form of its sulfoxide salt. In the present invention it is preferably in the non-salt form, i.e. as perphenazine.

Thioridazine is an antipsychotic drug belonging to the phenothiazine drug group. It is also known as 10-{2-[(RS)-1-methylpiperidin-2-yl]ethyl}-2-methylsulfanylphenothiazine or 10-[2-(1-methyl-2-piperidinyl)ethyl]-2-(methylthio)-10H-phenothiazine, and has the following chemical structure:

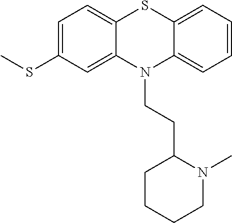

Thioridazine may be used in the form of its hydrochloride salt. In the present invention it is preferably in the salt form, i.e. as thioridazine hydrochloride.

Trifluoperazine is an antipsychotic drug of the phenothiazine chemical class. It is also known as 10-[3-(4-methylpiperazin-1-yl)propyl]-2-(trifluoromethyl)-10H-phenothiazine and has the following chemical structure:

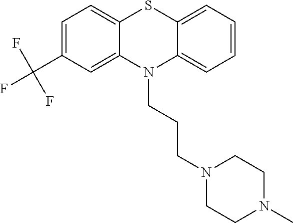 [SMILES: CN1CCN(CCCN2c3ccccc3Sc3ccc(C(F)(F)F)cc32)CC1]

Trifluoperazine may be used in the form of its hydrochloride salt or its dihydrochloride salt. In the present invention it is preferably in the hydrochloride salt form, i.e. as trifluoperazine hydrochloride.

Edetate (or ethylenediaminetetraacetic acid (EDTA)) is an aminopolycarboxylic acid which is widely used to dissolve limescale. It is also known as 2-({2-[bis(carboxymethyl)amino]ethyl}(carboxymethyl)amino)acetic acid and has the following chemical structure:

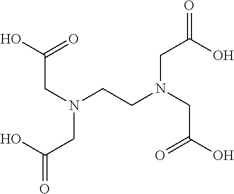 [SMILES: O=C(O)CN(CCN(CC(=O)O)CC(=O)O)CC(=O)O]

Edetate (or EDTA) may be used in the form of many salts and/or hydrates, including disodium edetate, edetate disodium, edetate calcium disodium, sodium calcium edetate, EDTA disodium salt dehydrate, EDTA tetrasodium salt dehydrate, EDTA ferric sodium salt, EDTA iron(III) sodium salt or EDTA iron (III) sodium salt hydrate. In the present invention it is preferably used as edetate disodium.

Compounds for use according to the invention may be administered as the raw material but the active ingredients are preferably provided in the form of pharmaceutical compositions. The active ingredients may be used either as separate formulations or as a single combined formulation. When combined in the same formulation it will be appreciated that the two compounds must be stable and compatible with each other and the other components of the formulation.

Formulations of the invention include those suitable for oral, parenteral (including subcutaneous e.g. by injection or by depot tablet, intrathecal, intramuscular e.g. by depot and intravenous), and rectal or in a form suitable for administration by inhalation or insufflation administration. The most suitable route of administration may depend upon the condition and disorder of the patient. Preferably, the compositions of the invention are formulated for parenteral administration. More preferably the compositions of the invention are formulated for intravenous or intramuscular administration.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

When formulated with excipients, the active ingredients may be present in a concentration from 0.1 to 99.5% (such as from 0.5 to 95%) by weight of the total mixture; conveniently from 30 to 95% for tablets and capsules and 0.01 to 50% (such as from 3 to 50%) for liquid preparations.

Formulations suitable for oral administration may be presented as discrete units such as capsules, cachets or tablets (e.g. chewable tablets in particular for pediatric administration), each containing a predetermined amount of active ingredient; as powder or granules; as a solution or suspension in an aqueous liquid or non-aqueous liquid; or as an oil-in-water liquid emulsion or water-in-oil liquid emulsion. The active ingredients may also be presented a bolus, electuary or paste.

A tablet may be made by compression or molding, optionally with one or more excipients. Compressed tablets may be prepared by compressing in a suitable machine the active ingredient in a free-flowing form such as a powder or granules, optionally mixed with other conventional excipients such as binding agents (e.g. syrup, acacia, gelatin, sorbitol, tragacanth, mucilage of starch, polyvinylpyrrolidone and/or hydroxymethyl cellulose), fillers (e.g. lactose, sugar, microcrystalline cellulose, maize-starch, calcium phosphate and/or sorbitol), lubricants (e.g. magnesium stearate, stearic acid, talc, polyethylene glycol and/or silica), disintegrants (e.g. potato starch, croscarmellose sodium and/or sodium starch glycolate) and wetting agents (e.g. sodium lauryl sulphate). Molded tablets may be made by molding in a suitable machine a mixture of the powdered active ingredient with an inert liquid diluent. The tablets may be optionally coated or scored and may be formulated so as to provide controlled release (e.g. delayed, sustained, or pulsed release, or a combination of immediate release and controlled release) of the active ingredients.

Alternatively, the active ingredients may be incorporated into oral liquid preparations such as aqueous or oily suspensions, solutions, emulsions, syrups or elixirs. Formulations containing the active ingredients may also be presented as a dry product for constitution with water or another suitable vehicle before use. Such liquid preparations may contain conventional additives such as suspending agents (e.g. sorbitol syrup, methyl cellulose, glucose/sugar syrup, gelatin, hydroxymethyl cellulose, carboxymethyl cellulose, aluminium stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

Combinations for use according to the invention may be presented in a pack or dispenser device which may contain one or more unit dosage forms containing the active ingredients. The pack may, e.g. comprise metal or plastic foil, such as a blister pack. Where the compositions are intended for administration as two separate compositions these may be presented in the form of a twin pack.

Pharmaceutical compositions may also be prescribed to the patient in “patient packs” containing the whole course of treatment in a single package, usually a blister pack. Patient packs have an advantage over traditional prescriptions, where a pharmacist divides a patients' supply of a pharmaceutical from a bulk supply, in that the patient always has access to the package insert contained in the patient pack, normally missing in traditional prescriptions. The inclusion of the package insert has been shown to improve patient compliance with the physician's instructions.

The compounds for use in the combination of the present invention are commercially available or can be prepared by synthesis methods known in the art. Imipenem, imipenem monohydrate, meropenem, meropenem trihydrate, ertapenem, ertapenem sodium, doripenem hydrate, doripenem monohydrate, biapenem, dichlorophen, chlorprothixene hydrochloride, perphenazine, perphenazine sulfoxide, thioridazine, thioridazine hydrochloride, trifluoperazine dihydrochloride, trifluoperazine hydrochloride and edetate disodium are for example available from Sigma-Aldrich®.

Suitable dosages and formulations are known in the art. For example, a suitable dosage and formulation for perphenazine is described in the product label for Fentazin which can be found at https://www.medicines.org.uk/emc/medicine/22596. A suitable dosage and formulation for trifluoperazine hydrochloride is described in the product label for Trifluoperazine either as 1 mg tablets, 1 mg/5 ml syrup, 5 mg tablets or 5 mg/5 ml oral solution which can be found at https://www.medicines.org.uk/emc/medicine/22564; https://www.medicines.org.uk/emc/medicine/22934; https://www.medicines.org.uk/emc/medicine/23635; and https://www.medicines.org/uk/emc/medicine/7909.

Suitable dosages and formulations for the administration of imipenem are described in the product label for imipenem/cilastatin 500 mg/500 mg powder for solution for infusion which can be found at https://www.medicines.org.uk/emc/medicine/24538; and the product label for Primaxin IV injection which can be found at https://www.medicines.org.uk/emc/medicine/7456. Suitable dosages and formulations for the administration of meropenem are described in the product label for Meronem IV which can be found at https://www.medicines.org.uk/emc/medicine/11215; or in the product label for Meropenem 1 g Powder for Solution for Injection or Infusion found at https://www.medicines.org.uk/emc/medicine/24151. Meropenem is administered intravenously. It is supplied as a white crystalline powder to be dissolved in 5% monobasic potassium phosphate solution. Suitable dosages and formulations for the administration of ertapenem are described in the product label for INVANZ® 1 g powder for concentrate for solution for infusion which can be found at https://www.medicines.org.uk/emc/medicine/10421.

The administration of the combination of the invention by means of a single patient pack, or patients packs of each composition, including a package insert directing the patient to the correct use of the invention is a desirable feature of this invention.

According to a further embodiment of the present invention there is provided a patient pack comprising at least one active ingredient of the combination according to the invention and an information insert containing directions on the use of the combination of the invention.

In another embodiment of the invention, there is provided a double pack comprising in association for separate administration, an antimicrobial agent, preferably having biological activity against clinically latent microorganisms, and one or more of the compounds disclosed herein preferably having biological activity against clinically latent microorganisms.

The amount of active ingredients required for use in treatment will vary with the nature of the condition being treated and the age and condition of the patient, and will ultimately be at the discretion of the attendant physician or veterinarian. In general however, doses employed for adult human treatment will typically be in the range of 0.02 to 5000 mg per day, preferably 1 to 1500 mg per day. The desired dose may conveniently be presented in a single dose or as divided doses administered at appropriate intervals, e.g. as two, three, four or more sub-doses per day.

### Biological Tests

Test procedures that may be employed to determine the biological (e.g. bactericidal or antimicrobial) activity of the active ingredients include those known to persons skilled in the art for determining:
(a) bactericidal activity against clinically latent bacteria; and
(b) antimicrobial activity against log phase bacteria.

In relation to (a) above, methods for determining activity against clinically latent bacteria include a determination, under conditions known to those skilled in the art (such as those described in Nature Reviews, Drug Discovery 1, 895-910 (2002), the disclosures of which are hereby incorporated by reference), of Minimum Stationary-cidal Concentration (“MSC”) or Minimum Dormicidal Concentration (“MDC”) for a test compound.

By way of example, WO2000028074 describes a suitable method of screening compounds to determine their ability to kill clinically latent microorganisms. A typical method may include the following steps:
(1) growing a bacterial culture to stationery phase;
(2) treating the stationery phase culture with one or more antimicrobial agents at a concentration and or time sufficient to kill growing bacteria, thereby selecting a phenotypically resistant sub-population;
(3) incubating a sample of the phenotypically resistant subpopulation with one or more test compounds or agents; and
(4) assessing any antimicrobial effects against the phenotypically resistant subpopulation.

According to this method, the phenotypically resistant sub-population may be seen as representative of clinically latent bacteria which remain metabolically active in vivo and which can result in relapse or onset of disease.

In relation to (b) above, methods for determining activity against log phase bacteria include a determination, under standard conditions (i.e. conditions known to those skilled in the art, such as those described in WO 2005014585, the disclosures of which document are hereby incorporated by reference), of Minimum Inhibitory Concentration (“MIC”) or Minimum Bactericidal Concentration (“MBC”) for a test compound. Specific examples of such methods are described below.

### EXAMPLES

### Example 1: In Vitro Synergistic Effect of Dichlorophen and Meropenem Trihydrate Against Log Phase NDM-1 Klebsiella pneumoniae Subsp. Pneumoniae Using the Chequerboard Method

The chequerboard method used in Example 1 followed the protocols detailed in Antimicrob Chemo (2013) 68, 374-384. Log phase growth of NDM-1 Klebsiella pneumoniae subsp. pneumoniae was carried out as described in the art. Dichlorophen and meropenem trihydrate were obtained from commercially available sources (e.g. Sigma-Aldrich® UK). The effects of the combination of the present invention were examined by calculating the fractional inhibitory concentration index (FICI) of each combination, as follows:

(MIC of drug A, tested in combination)/(MIC of drug A, tested alone)+(MIC of drug B, tested in combination)/(MIC of drug B, tested alone).

The interaction of the combination was defined as showing synergy if the FICI was ≤0.5, no interaction if the FICI was >0.5 but <2 and antagonism if the FICI was ≥2.

BAA2472

| | | Meropenem | | | | | | | | | | | | |
|---|---|---|---|---|---|---|---|---|---|---|---|---|---|---|
| | | 1667 | 833.5 | 416.75 | 208.375 | 104.1875 | 52.09375 | 26.04688 | 13.02344 | 6.511719 | 3.255859 | 1.62793 | 0 | |
| Di- | 256 | 0.17 | 0.10 | 0.08 | 0.08 | 0.08 | 0.07 | 0.07 | 0.07 | 0.07 | 0.07 | 0.07 | 0.08 | 0.38 |
| chloro- | 128 | 0.09 | 0.07 | 0.06 | 0.06 | 0.06 | 0.06 | 0.06 | 0.06 | 0.06 | 0.06 | 0.06 | 0.06 | |
| phen | 64 | 0.07 | 0.06 | 0.06 | 0.06 | 0.23 | 0.22 | 0.26 | 0.26 | 0.25 | 0.25 | 0.23 | 0.29 | |
| | 32 | 0.06 | 0.06 | 0.06 | 0.19 | 0.20 | 0.20 | 0.23 | 0.22 | 0.23 | 0.20 | 0.22 | 0.25 | |
| | 16 | 0.06 | 0.05 | 0.06 | 0.24 | 0.24 | 0.21 | 0.24 | 0.27 | 0.28 | 0.28 | 0.28 | 0.31 | |
| | 8 | 0.06 | 0.05 | 0.15 | 0.23 | 0.32 | 0.24 | 0.38 | 0.49 | 0.42 | 0.41 | 0.41 | 0.47 | |
| | 4 | 0.06 | 0.05 | 0.13 | 0.22 | 0.31 | 0.25 | 0.50 | 0.49 | 0.47 | 0.45 | 0.48 | 0.53 | |
| | 0 | 0.06 | 0.05 | 0.05 | 0.26 | 0.37 | 0.48 | 0.56 | 0.57 | 0.57 | 0.57 | 0.56 | 0.54 | |

BAA2470

| | | Meropenem | | | | | | | | | | | | |
|---|---|---|---|---|---|---|---|---|---|---|---|---|---|---|
| | | 256 | 128 | 64 | 32 | 16 | 8 | 4 | 2 | 1 | 0.5 | 0.25 | 0 | FIC index |
| Di- | 256 | 0.09 | 0.08 | 0.07 | 0.07 | 0.07 | 0.07 | 0.07 | 0.07 | 0.07 | 0.07 | 0.07 | 0.08 | 0.5 |
| chloro- | 128 | 0.06 | 0.06 | 0.05 | 0.05 | 0.05 | 0.05 | 0.05 | 0.05 | 0.05 | 0.05 | 0.05 | 0.05 | |
| phen | 64 | 0.05 | 0.05 | 0.10 | 0.17 | 0.19 | 0.20 | 0.21 | 0.22 | 0.18 | 0.20 | 0.16 | 0.22 | |
| | 32 | 0.05 | 0.08 | 0.17 | 0.17 | 0.19 | 0.21 | 0.21 | 0.22 | 0.22 | 0.20 | 0.20 | 0.22 | |
| | 16 | 0.05 | 0.13 | 0.16 | 0.17 | 0.20 | 0.23 | 0.23 | 0.25 | 0.24 | 0.23 | 0.19 | 0.23 | |
| | 8 | 0.05 | 0.14 | 0.16 | 0.22 | 0.30 | 0.31 | 0.29 | 0.31 | 0.29 | 0.27 | 0.24 | 0.29 | |
| | 4 | 0.04 | 0.13 | 0.16 | 0.23 | 0.27 | 0.31 | 0.32 | 0.31 | 0.31 | 0.30 | 0.30 | 0.41 | |
| | 0 | 0.04 | 0.24 | 0.30 | 0.33 | 0.42 | 0.46 | 0.44 | 0.45 | 0.44 | 0.39 | 0.38 | 0.56 | |

The FICI (FIC index) was equal to 0.38 and 0.5 indicating that dichlorophen and meropenem trihydrate have a synergistic effect when used in combination against log phase NDM-1 Klebsiella pneumoniae subsp. pneumoniae.

### Example 2: In Vitro Synergistic Effect of Thioridazine Hydrochloride in Combination with Meropenem Trihydrate Against Log Phase NDM-1 Escherichia coli

Log phase growth of NDM-1 Escherichia coli was carried out as described in the art. Thioridazine hydrochloride and meropenem trihydrate were obtained from commercial sources (e.g. Sigma Aldrich® UK). The effect of the combination of the present invention was examined by using the chequerboard method and calculating the fractional inhibitory concentration index (FICI) of each combination in the same manner as for Example 1. The chequerboard data is shown below:

BAA2471

| | | Meropenem | | | | | | | | | | | | |
|---|---|---|---|---|---|---|---|---|---|---|---|---|---|---|
| | | 1667 | 833.5 | 416.75 | 208.375 | 104.1875 | 52.09375 | 26.04688 | 13.02344 | 6.511719 | 3.255859 | 1.62793 | 0 | |
| Thiorida- | 256 | 0.06 | 0.05 | 0.05 | 0.05 | 0.06 | 0.06 | 0.07 | 0.08 | 0.08 | 0.08 | 0.09 | 0.09 | 0.28 |
| zine | 128 | 0.06 | 0.05 | 0.05 | 0.04 | 0.04 | 0.04 | 0.04 | 0.05 | 0.05 | 0.05 | 0.05 | 0.05 | |
| Hydro- | 64 | 0.06 | 0.05 | 0.05 | 0.04 | 0.04 | 0.21 | 0.20 | 0.16 | 0.18 | 0.19 | 0.19 | 0.18 | |
| chloride | 32 | 0.06 | 0.05 | 0.04 | 0.04 | 0.47 | 0.50 | 0.50 | 0.50 | 0.54 | 0.53 | 0.53 | 0.53 | |
| | 16 | 0.06 | 0.05 | 0.04 | 0.04 | 0.51 | 0.55 | 0.58 | 0.58 | 0.61 | 0.65 | 0.60 | 0.57 | |
| | 8 | 0.06 | 0.05 | 0.05 | 0.04 | 0.52 | 0.56 | 0.61 | 0.60 | 0.61 | 0.64 | 0.63 | 0.56 | |
| | 4 | 0.06 | 0.05 | 0.04 | 0.04 | 0.50 | 0.56 | 0.61 | 0.59 | 0.64 | 0.65 | 0.68 | 0.58 | |
| | 0 | 0.06 | 0.05 | 0.05 | 0.51 | 0.58 | 0.62 | 0.62 | 0.63 | 0.65 | 0.64 | 0.66 | 0.61 | |

The FICI was equal to 0.28 indicating that thioridazine hydrochloride and meropenem trihydrate exhibited a synergistic effect against log phase NDM-1 E. coli.

### Example 3: In Vitro Synergistic Effect of Trifluoperazine Hydrochloride and Meropenem Trihydrate Against Log Phase NDM-1 Escherichia coli Using the Chequerboard Method

Log phase growth of NDM-1 Escherichia coli was carried out as described in the art. Trifluoperazine hydrochloride and meropenem trihydrate were obtained from commercial sources (e.g. Sigma Aldrich® UK). The effects of each combination of the present invention were examined by using the chequerboard method and calculating the fractional inhibitory concentration index (FICI) of each combination in the same manner as for Example 1. The chequerboard data is shown below:

BAA2471

| | | Meropenem | | | | | | | | | | | | |
|---|---|---|---|---|---|---|---|---|---|---|---|---|---|---|
| | | 1667 | 833.5 | 416.75 | 208.375 | 104.1875 | 52.09375 | 26.04688 | 13.02344 | 6.511719 | 3.255859 | 1.62793 | 0 | |
| trifluo- | 256 | 0.07 | 0.06 | 0.08 | 0.09 | 0.10 | 0.10 | 0.10 | 0.09 | 0.09 | 0.09 | 0.10 | 0.13 | 0.25 |
| perazine | 128 | 0.06 | 0.05 | 0.05 | 0.05 | 0.05 | 0.05 | 0.05 | 0.05 | 0.05 | 0.05 | 0.05 | 0.05 | |
| hydro- | 64 | 0.06 | 0.05 | 0.05 | 0.04 | 0.04 | 0.18 | 0.17 | 0.17 | 0.17 | 0.17 | 0.17 | 0.18 | |
| chloride | 32 | 0.06 | 0.05 | 0.13 | 0.35 | 0.48 | 0.54 | 0.56 | 0.54 | 0.58 | 0.57 | 0.55 | 0.52 | |
| | 16 | 0.06 | 0.05 | 0.04 | 0.43 | 0.51 | 0.61 | 0.61 | 0.60 | 0.65 | 0.63 | 0.62 | 0.56 | |
| | 8 | 0.06 | 0.05 | 0.04 | 0.42 | 0.58 | 0.60 | 0.63 | 0.63 | 0.65 | 0.66 | 0.66 | 0.57 | |
| | 4 | 0.06 | 0.05 | 0.04 | 0.39 | 0.54 | 0.61 | 0.65 | 0.66 | 0.63 | 0.66 | 0.68 | 0.57 | |
| | 0 | 0.06 | 0.05 | 0.05 | 0.50 | 0.59 | 0.67 | 0.64 | 0.66 | 0.62 | 0.66 | 0.68 | 0.58 | |

The FICI was equal to 0.25 indicating that trifluoperazine hydrochloride and meropenem trihydrate exhibited a synergistic effect against log phase NDM-1 E. coli.

### Example 4: In Vitro Synergistic Effect of Chlorprothixene Hydrochloride and Meropenem Trihydrate Against Log Phase NDM-1 Klebsiella pneumoniae Subsp. Pneumoniae Using the Chequerboard Method

Log phase growth of NDM-1 K. pneumoniae subsp. pneumoniae was carried out as described in the art. Chlorprothixene hydrochloride and meropenem trihydrate were obtained from commercial sources (e.g. Sigma Aldrich® UK). The effects of each combination of the present invention were examined by using the chequerboard method and calculating the fractional inhibitory concentration index (FICI) of each combination in the same manner as for Example 1. The chequerboard data is shown below:

BAA2472

| | | Meropenem | | | | | | | | | | | | |
|---|---|---|---|---|---|---|---|---|---|---|---|---|---|---|
| | | 1667 | 833.5 | 416.75 | 208.375 | 104.1875 | 52.09375 | 26.04688 | 13.02344 | 6.511719 | 3.255859 | 1.62793 | 0 | |
| chlorpro- | 256 | 0.16 | 0.17 | 0.18 | 0.15 | 0.15 | 0.13 | 0.50 | 0.53 | 0.54 | 0.49 | 0.50 | 0.51 | 0.28 |
| thixene | 128 | 0.06 | 0.05 | 0.05 | 0.05 | 0.05 | 0.24 | 0.24 | 0.25 | 0.26 | 0.25 | 0.27 | 0.36 | |
| hydro- | 64 | 0.05 | 0.05 | 0.04 | 0.04 | 0.13 | 0.18 | 0.17 | 0.17 | 0.17 | 0.20 | 0.23 | 0.31 | |
| chloride | 32 | 0.06 | 0.04 | 0.04 | 0.04 | 0.28 | 0.32 | 0.21 | 0.20 | 0.27 | 0.32 | 0.40 | 0.48 | |
| | 16 | 0.06 | 0.04 | 0.04 | 0.05 | 0.25 | 0.28 | 0.31 | 0.50 | 0.49 | 0.51 | 0.62 | 0.68 | |
| | 8 | 0.05 | 0.04 | 0.04 | 0.04 | 0.27 | 0.37 | 0.33 | 0.49 | 0.48 | 0.53 | 0.61 | 0.66 | |
| | 4 | 0.05 | 0.04 | 0.04 | 0.21 | 0.43 | 0.40 | 0.47 | 0.54 | 0.54 | 0.58 | 0.63 | 0.67 | |
| | 0 | 0.06 | 0.05 | 0.05 | 0.53 | 0.60 | 0.62 | 0.72 | 0.76 | 0.74 | 0.76 | 0.75 | 0.63 | |

The FICI was equal to 0.28 indicating that chlorprothixene hydrochloride and meropenem trihydrate exhibited a synergistic effect against log phase NDM-1 K.pneumoniae subsp. pneumoniae.

### Example 5: In Vitro Synergistic Effect of Perphenazine and Meropenem Trihydrate Against Log Phase NDM-1 E. coli Using the Chequerboard Method

Log phase growth of NDM-1 E. coli was carried out as described in the art. Perphenazine and meropenem trihydrate were obtained from commercial sources (e.g. Sigma Aldrich® UK). The effects of each combination of the present invention were examined by using the chequerboard method and calculating the fractional inhibitory concentration index (FICI) of each combination in the same manner as for Example 1. The chequerboard data is shown below:

BAA2471

| | | Meropenem | | | | | | | | | | | | |
|---|---|---|---|---|---|---|---|---|---|---|---|---|---|---|
| | | 1667 | 833.5 | 416.75 | 208.375 | 104.1875 | 52.09375 | 26.04688 | 13.02344 | 6.511719 | 3.255859 | 1.62793 | 0 | |
| perphe- | 256 | 0.11 | 0.16 | 0.30 | 0.40 | 0.41 | 0.51 | 0.56 | 0.60 | 0.66 | 0.72 | 0.65 | 0.65 | 0.27 |
| nazine | 128 | 0.05 | 0.05 | 0.05 | 0.05 | 0.05 | 0.07 | 0.08 | 0.10 | 0.11 | 0.11 | 0.13 | 0.20 | |
| | 64 | 0.05 | 0.05 | 0.04 | 0.04 | 0.24 | 0.19 | 0.16 | 0.26 | 0.31 | 0.32 | 0.32 | 0.28 | |
| | 32 | 0.05 | 0.46 | 0.40 | 0.41 | 0.54 | 0.58 | 0.57 | 0.57 | 0.61 | 0.61 | 0.67 | 0.51 | |
| | 16 | 0.05 | 0.51 | 0.46 | 0.45 | 0.64 | 0.67 | 0.69 | 0.69 | 0.71 | 0.79 | 0.75 | 0.60 | |
| | 8 | 0.05 | 0.52 | 0.54 | 0.47 | 0.67 | 0.72 | 0.73 | 0.74 | 0.74 | 0.73 | 0.74 | 0.59 | |
| | 4 | 0.05 | 0.53 | 0.49 | 0.46 | 0.66 | 0.69 | 0.72 | 0.71 | 0.73 | 0.72 | 0.78 | 0.56 | |
| | 0 | 0.05 | 0.49 | 0.44 | 0.41 | 0.64 | 0.71 | 0.72 | 0.68 | 0.70 | 0.64 | 0.68 | 0.62 | |

The FICI was equal to 0.27 indicating that perphenazine and meropenem trihydrate exhibited a synergistic effect against log phase NDM-1 E. coli.

### Example 6: In Vitro Synergistic Effect of Thioridazine Hydrochloride and Meropenem Trihydrate Against Log Phase NDM-1 Klebsiella pneumoniae Subsp. Pneumoniae Using the Chequerboard Method

Log phase growth of NDM-1 K. pneumoniae subsp. pneumoniae was carried out as described in the art. Thioridazine hydrochloride and meropenem trihydrate were obtained from commercial sources (e.g. Sigma Aldrich® UK). The effects of each combination of the present invention were examined by using the chequerboard method and calculating the fractional inhibitory concentration index (FICI) of each combination in the same manner as for Example 1. The chequerboard data is shown below:

BAA2470

| | | Meropenem | | | | | | | | | | | | |
|---|---|---|---|---|---|---|---|---|---|---|---|---|---|---|
| | | 1667 | 833.5 | 416.75 | 208.375 | 104.1875 | 52.09375 | 26.04688 | 13.02344 | 6.511719 | 3.255859 | 1.62793 | 0 | |
| Thiorida- | 256 | 0.06 | 0.07 | 0.08 | 0.09 | 0.08 | 0.08 | 0.07 | 0.07 | 0.08 | 0.13 | 0.41 | 0.56 | 0.19 |
| zine | 128 | 0.06 | 0.05 | 0.05 | 0.05 | 0.05 | 0.05 | 0.05 | 0.08 | 0.10 | 0.12 | 0.15 | 0.28 | |
| Hydro- | 64 | 0.06 | 0.05 | 0.05 | 0.05 | 0.05 | 0.04 | 0.14 | 0.13 | 0.14 | 0.13 | 0.14 | 0.24 | |
| chloride | 32 | 0.06 | 0.05 | 0.04 | 0.04 | 0.04 | 0.05 | 0.18 | 0.19 | 0.19 | 0.16 | 0.20 | 0.32 | |
| | 16 | 0.06 | 0.05 | 0.04 | 0.04 | 0.04 | 0.24 | 0.77 | 0.20 | 0.21 | 0.20 | 0.35 | 0.57 | |
| | 8 | 0.06 | 0.05 | 0.04 | 0.04 | 0.04 | 0.16 | 0.22 | 0.19 | 0.20 | 0.22 | 0.39 | 0.56 | |
| | 4 | 0.06 | 0.05 | 0.04 | 0.04 | 0.04 | 0.21 | 0.24 | 0.24 | 0.27 | 0.27 | 0.40 | 0.60 | |
| | 0 | 0.06 | 0.05 | 0.05 | 0.05 | 0.05 | 0.22 | 0.39 | 0.40 | 0.41 | 0.44 | 0.53 | 0.58 | |

The FICI was equal to 0.19 indicating that thioridazine hydrochloride and meropenem trihydrate exhibited a synergistic effect against log phase NDM-1 K. pneumoniae subsp. pneumoniae.

### Example 7: In Vitro Synergistic Effect of Edetate Disodium and Meropenem Trihydrate Against Log Phase NDM-1 Klebsiella pneumoniae Subsp. Pneumoniae Using the Chequerboard Method

Log phase growth of NDM-1 K. pneumoniae subsp. pneumoniae was carried out as described in the art. Edetate disodium and meropenem trihydrate were obtained from commercial sources (e.g. Sigma Aldrich® UK). The effects of each combination of the present invention were examined by using the chequerboard method and calculating the fractional inhibitory concentration index (FICI) of each combination in the same manner as for Example 1. The chequerboard data is shown below:

BAA2472

| | | Meropenem | | | | | | | | | | | | |
|---|---|---|---|---|---|---|---|---|---|---|---|---|---|---|
| | | 256 | 128 | 64 | 32 | 16 | 8 | 4 | 2 | 1 | 0.5 | 0.25 | 0 | |
| edetate | 256 | 0.04 | 0.04 | 0.04 | 0.04 | 0.04 | 0.04 | 0.04 | 0.04 | 0.04 | 0.11 | 0.21 | 0.34 | 0.07 |
| disodium | 128 | 0.04 | 0.04 | 0.04 | 0.04 | 0.04 | 0.04 | 0.04 | 0.13 | 0.05 | 0.10 | 0.17 | 0.33 | |
| | 64 | 0.04 | 0.04 | 0.04 | 0.04 | 0.04 | 0.22 | 0.04 | 0.25 | 0.23 | 0.20 | 0.17 | 0.33 | |
| | 32 | 0.04 | 0.04 | 0.04 | 0.07 | 0.22 | 0.25 | 0.25 | 0.25 | 0.25 | 0.22 | 0.21 | 0.33 | |
| | 16 | 0.04 | 0.04 | 0.12 | 0.17 | 0.25 | 0.27 | 0.27 | 0.25 | 0.24 | 0.22 | 0.21 | 0.35 | |
| | 8 | 0.04 | 0.17 | 0.15 | 0.17 | 0.24 | 0.25 | 0.25 | 0.29 | 0.25 | 0.24 | 0.23 | 0.35 | |
| | 4 | 0.04 | 0.18 | 0.18 | 0.16 | 0.21 | 0.24 | 0.24 | 0.24 | 0.24 | 0.21 | 0.22 | 0.36 | |
| | 0 | 0.04 | 0.24 | 0.28 | 0.30 | 0.34 | 0.35 | 0.35 | 0.36 | 0.43 | 0.34 | 0.44 | 0.39 | |

The FICI was equal to 0.07 indicating that edetate disodium and meropenem trihydrate exhibited a synergistic effect against log phase NDM-1 K. pneumoniae subsp. pneumoniae.

### Example 8: In Vitro Synergistic Effect of Dichlorophen in Combination with Meropenem Trihydrate Against Log Phase NDM-1 Klebsiella Pneumoniae

The objective of this example was to test the synergistic effect of dichlorophen and meropenem trihydrate in combination against log phase NDM-1 Klebsiella Pneumoniae (NCTC13443) by time-kill methods over a time period of 24 hours. As described hereinabove, time-kill methods are another accepted test for ascertaining the presence or absence of synergy, and involve comparing the dynamic effect of a drug combination with each drug alone when assessing the effect on bacterial log or stationary-growth over time. The results can either show that the drug combination is synergistic, additive or antagonistic. Such a result is not predictable from the activity of either drug alone or in combination with another agent.

### Materials and Methods

1. Bacterial strain used: NCTC13443 strain of Klebsiella Pneumoniae
2. Growth of bacteria: Log phase growth of NCTC13443 was carried out according to known methods in the art, e.g. SOP R-005-00 Log Phase Growth of Bacteria.
3. Compound preparation: dichlorophen and meropenem trihydrate were both obtained from commercial sources (e.g. Sigma-Aldrich® UK).
4. The overnight culture was diluted with nutrient broth (Oxoid) to 10⁷ CFU/ml and 280 μl and 290 μl of the culture was added to each combination well and drug respectively, to make the final concentration of 300 μl.
5. Incubation of the compounds with the bacterial suspension was carried out for 24 hours. At 0, 2, 4, 7 and 24 hours, CFU counts were performed to measure the kill effects of the drug combination.

### Results and Discussion

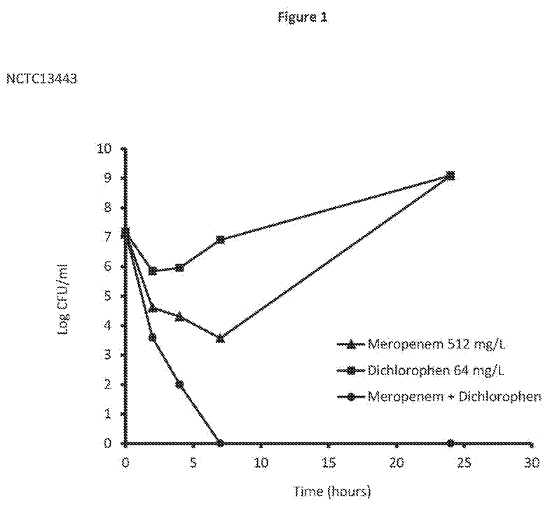

The results are shown in FIG. 1 where it can be seen that dichlorophen used alone at a concentration of 64 mg/L and meropenem trihydrate used alone at a concentration of 512 mg/L had little or no effect against log phase NDM-1 Klebsiella Pneumoniae. When used in combination, however, a significant synergistic effect can be seen: complete, long term kill of bacteria occurs at 7 hours. In view of the lack of activity for each of the drugs alone, the synergy seen for the combination of the claimed invention is a surprising and advantageous technical effect for the treatment of microbial infections.

1. A combination comprising: (i) at least one compound selected from dichlorophen, chlorprothixene, perphenazine, thioridazine, trifluoperazine and edetate or a pharmaceutically acceptable derivative thereof; and (ii) a carbapenem or a pharmaceutically acceptable derivative thereof.

2. The combination according to claim 1, wherein the carbapenem is selected from one or more of imipenem, meropenem, doripenem and ertapenem or a pharmaceutically acceptable derivative thereof.

3. The combination according to claim 1, wherein the carbapenem is meropenem or a pharmaceutically acceptable derivative thereof.

4. (canceled)

5. The method according to claim 16, wherein the microbial infection is a bacterial infection selected from at least one a gram-negative bacterial infection.

6. The method according to claim 5, wherein the bacterial infection is caused by E. coli or Klebs. pneumoniae.

7. The method according to claim 17, wherein the infection is caused by a drug-resistant strain of the bacteria.

8. (canceled)

9. (canceled)

10. The combination according to claim 1, wherein the combination is used to treat a microbial infection.

11. The combination according to claim 19, wherein the bacterial infection is a gram-negative bacterial infection caused by E. coli or Klebs. pneumoniae.

12. The combination according to claim 10, wherein the microbial infection is caused by a drug-resistant strain.

13. The combination according to claim 1 for use in the treatment of abscesses, asthma, bacilliary dysentry, bacterial conjunctivitis, bacterial keratitis, bacterial vaginosis, bone and joint infections, bronchitis (acute or chronic), brucellosis, burn wounds, cat scratch fever, cellulitis, chancroid, cholangitis, cholecystitis, cystic fibrosis, cystitis, nephritis, diffuse panbronchiolitis, dental caries, diseases of the upper respiratory tract, empymea, endocarditis, endometritis, enteric fever, enteritis, epididymitis, epiglottitis, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leptospirosis, listeriosis, liver abscesses, Lyme disease, lymphogranuloma venerium, mastitis, mastoiditis, meningitis and infections of the nervous system, non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, pleural effusion, pneumonia, postoperative wound infections, postoperative gas gangrene, prostatitis, pseudo-membranous colitis, psittacosis, pyelonephritis, Q fever, rat-bite fever, Ritter's disease, salmonellosis, salpingitis, septic arthritis, septic infections, septicameia, systemic infections, tonsillitis, trachoma, typhoid, urethritis, urinary tract infections, wound infections; or infections with, Escherichia coli, Klebs. pneumoniae, Klebs. oxytoca, Pr. mirabilis, Pr. rettgeri, Pr. vulgaris, Haemophilis influenzae, Enterococcus faecalis, Enterococcus faecium, and Enterobacter cloacae.

14. A pharmaceutical composition comprising: (a) at least one compound selected from dichlorophen, chlorprothixene, perphenazine, thioridazine, trifluoperazine and edetate or a pharmaceutically acceptable derivative thereof in combination with: (b) a carbapenem or a pharmaceutically acceptable derivative thereof; and (c) a pharmaceutically acceptable adjuvant, diluent or carrier.

15. A product comprising: (i) at least one compound selected from dichlorophen, chlorprothixene, perphenazine, thioridazine, trifluoperazine and edetate or a pharmaceutically acceptable derivative thereof; and (ii) and a carbapenem or a pharmaceutically acceptable derivative thereof, wherein the product is a combined preparation for the simultaneous, separate or sequential use in treating a microbial infection.

16. A method of treating a microbial infection, the method comprising the step of administering at least one therapeutic amount of the combination according to claim 1 to a desired subject.

17. The method of claim 16, wherein the method is used to treat a microbial infection.

18. The method of claim 16, wherein the method is used in the treatment of abscesses, asthma, bacilliary dysentry, bacterial conjunctivitis, bacterial keratitis, bacterial vaginosis, bone and joint infections, bronchitis (acute or chronic), brucellosis, burn wounds, cat scratch fever, cellulitis, chancroid, cholangitis, cholecystitis, cystic fibrosis, cystitis, nephritis, diffuse panbronchiolitis, dental caries, diseases of the upper respiratory tract, empymea, endocarditis, endometritis, enteric fever, enteritis, epididymitis, epiglottitis, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leptospirosis, listeriosis, liver abscesses, Lyme disease, lymphogranuloma venerium, mastitis, mastoiditis, meningitis and infections of the nervous system, non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, pleural effusion, pneumonia, postoperative wound infections, postoperative gas gangrene, prostatitis, pseudo-membranous colitis, psittacosis, pyelonephritis, Q fever, rat-bite fever, Ritter's disease, salmonellosis, salpingitis, septic arthritis, septic infections, septicameia, systemic infections, tonsillitis, trachoma, typhoid, urethritis, urinary tract infections, wound infections; or infections with, Escherichia coli, Klebs. pneumoniae, Klebs. oxytoca, Pr. mirabilis, Pr. rettgeri, Pr. vulgaris, Haemophilis influenzae, Enterococcus faecalis, Enterococcus faecium, and Enterobacter cloacae.

19. The combination according to claim 10, wherein the microbial infection is a bacterial infection.

20. The combination according to claim 19, wherein the microbial infection is a gram-negative bacterial infection.
